(19) United States
(12) Patent Application Publication
(10) Pub. No.: US 2002/0115612 A1
(43) Pub. Date: Aug. 22, 2002

(54) Poly (N-substituted glycines) with nucleotide base substituents

(75) Inventors: Ronald N. Zuckermann, El Cerrito, CA (US); Janice M. Kerr, Oakland, CA (US); Stephen Brian Henry Kent, La Jolla, CA (US)

Correspondence Address: Chiron Corporation, Intellectual Property - R440, 4560 Horton Street, Emeryville, CA, 94608-2916, US

(73) Assignee: Chiron Corporation

(21) Appl. No.: 10/071,577

(22) Filed: Feb. 8, 2002

Related U.S. Application Data
(62) Division of application No. 09/573,700, filed on May 19, 2000, now abandoned.
Application No. 09/573,700 is a division of application No. 08/454,511, filed on May 30, 1995, now abandoned.
Application No. 08/454,511 is a continuation of application No. 07/950,853, filed on Sep. 24, 1992, now abandoned.

(51) Int. Cl. A61K 38/16; C07K 14/00

(52) U.S. Cl. 514/12; 530/350

(57) ABSTRACT

An automated solid-phase method for the synthesis of poly (N-substituted glycines) (referred to herein as poly NSGs) taught here can be used to obtain poly NSGs of potential therapeutic interest which poly NSGs can have a wide variety of side chain substituents. Each N-substituted glycine monomer is assembled from two “sub-monomers” directly on the solid support. Each cycle of monomer addition consists of two steps: (1) acylation of a resin-bound secondary amine with an agent such as a haloacetic acid, and (2) introduction of the side-chain by nucleophilic displacement of the halogen (as a resin-bound α-haloacetamide) with an excess of primary amine. The efficient synthesis of a wide variety of oligomeric NSGs using automated synthesis technology, as presented here, makes these polymers attractive candidates for the generation and rapid screening of diverse peptidomimetic libraries. The oligomers of N-substituted glycines (i.e. poly NSGs) disclosed here provide a new class of polymers not found in nature, but which are synthetically accessible and have been shown to possess significant biological activity and proteolytic stability.

### FIELD OF THE INVENTION

The present invention relates generally to chemical synthesis technologies. More particularly, the present invention relates to the synthesis of peptide-like compounds in the form of poly (N-substituted glycines) (referred to herein as poly NSGs) using solid-phase synthesis methodology.

### BACKGROUND OF THE INVENTION

Standard methods analogous to classical solid-phase methods for peptide synthesis could be applied for the synthesis of NSGs. In accordance with such methods, the carboxylate of N,α-Fmoc-protected (and side-chain protected) NSGs would be activated and then coupled to a resin-bound amino group. The Fmoc group is then removed followed by addition of the next monomer. Thus, oligomeric NSGs could be prepared as condensation homopolymers of N-substituted glycine. Such an approach is not desirable due to the time and cost of preparing suitable quantities of a diverse set of protected N-substituted glycine monomers. Adding and removing the Fmoc or other protective groups is time consuming and inefficient.

### SUMMARY OF THE INVENTION

Synthesis methodology is disclosed whereby each N-substituted glycine monomer is assembled from two “sub-monomers” directly on a solid support. Thus, oligomeric N-substituted glycines (poly NSGs) are prepared as alternating condensation copolymers of a haloacetic acid and a primary amine. The direction of polymer synthesis with the sub-monomers occurs in the carboxy to amino direction. The solid-phase assembly of each monomer—and concurrent polymer formation—eliminates the need for N,α-protected monomers. Only reactive side-chain functionalities need be protected. Moreover, each sub-monomer is simpler in structure (many are commercially available), which dramatically reduces the time and cost required for poly NSG synthesis.

A primary object of the present invention is to disclose a method of synthesizing poly (N-substituted glycines).

Another object of the invention is to disclose solid-phase methodology for synthesizing polymers of N-substituted glycines which polymers can have a wide variety of side chain substituents.

An advantage of the present invention is that the methodology can be carried out more efficiently than conventional synthesis using solid-phase methodologies.

Another advantage of the present invention is that the methodology eliminates the need for N,α-protected monomers.

Yet another advantage of the present invention is that each sub-monomer of the polymer has a simple structure allowing for quick and efficient synthesis.

A feature of the present invention is that only the reactive side-chain functionalities need be protected or blocked during the synthesis.

Another feature of the present invention is that many of the sub-monomer components used in connection with the invention are commercially available.

These and other objects, advantages and features of the present invention will become apparent to those persons skilled in the art upon reading the details of the structure, synthesis and usage as more fully set forth below.

### DETAILED DESCRIPTION OF THE PREFERRED EMBODIMENTS

Before the present solid-phase synthesis methodology is disclosed and described, it is to be understood that this invention is not limited to the particular polymers, conditions and techniques described as such may, of course, vary. It is also to be understood that the terminology used herein is for the purpose of describing particular embodiments only, and is not intended to be limiting since the scope of the present invention will be limited only by the appended claims.

It must be noted that as used in this specification and the appended claims, the singular forms “a,” “and,” and “the” include plural referents unless the context clearly dictates otherwise. Thus, for example, reference to “a reactant” such as “a sub-monomer” include a plurality and/or mixture of such monomers, reference to “an N,α-protected monomer” includes a plurality of such monomers and reference to “the polymer” includes a plurality and mixtures of such polymers and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of disclosing and describing features of the invention for which the publications are cited in connection with.

An important aspect of the invention is an automated and highly efficient solid-phase method for synthesizing a specific type of polymer which is referred to herein as poly (N-substituted glycines) (hereinafter poly NSGs). The poly NSGS, which can be produced using the methodology of the present invention are not peptides. However, they can be designed so as to have closely related structural similarities (e.g., reactive sites) to naturally occurring peptides and proteins and as such are useful as potential therapeutic agents. The poly NSGs disclosed herein can be designed so as to have a wide variety of side chain substituents—including substituents normally found on natural amino acids and others not naturally occurring.

In accordance with the basic methodology, each N-substituted glycine monomer is assembled from two reactants which are referred to herein as sub-monomers directly on a solid support. Each monomer is produced by a synthesis cycle which is comprised of two steps. In accordance with the first step, acylation of a resin-bound secondary amine is carried out using a haloacetic acid. The second step involves the introduction of a side chain by nucleophilic displacement of the halogen by providing an excess of primary amine.

Another object of the invention is a method of producing poly NSGs also referred to herein as oligomeric-(N-substituted) glycines. The polyamide structures differ from polypeptides in that the side chains are substituted on the nitrogen rather than the α-carbon. The compounds of the invention have the following general structural formula I.

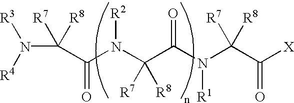

wherein R¹, R³ and R⁴ as well as each R² is independently any molecular moiety attachable to the nitrogen atom; R⁷ and R⁸ are independently each a moiety attached to a carbon atom including —H or an alkyl moiety containing 1 to 6 carbon atoms, and are preferably —CH₃ and more preferably —H. X is —NR⁵ [where R⁵₂ is as R² and is preferably H₂, H and an alkyl (1-6 carbons) or two lower alkyls] or —OR⁶ [where R⁶ is —H or a lower alkyl (1-6 carbons)] and n is an integer of from 1 to 2,000 preferably 2-100, more preferably 2-12 and most preferably 3-8. Although R¹, R², R³ and R⁴ may be any molecular moiety, examples of useful moieties (in particular for R²) include the side chain moieties present on a naturally occurring amino acid, i.e., —H of glycine; —CH₃ of alanine; —CH(CH₃)₂ of valine; —CH₂CH(CH₃)₂ of leucine; —CH(CH₃)CH₂CH₃ of isoleucine; —CH₂OH of serine; —CHOHCH₃ of threonine; —CH₂SH of cysteine; —CH₂CH₂SCH₃ of methionine; —CH₂-(phenyl) of phenylalanine; —CH₂-(phenyl)-OH of tyrosine; —CH₂-(indole group) of tryptophan; —CH₂COO⁻ of aspartic acid; —CH₂C(O)(NH₂) of aspargine; —CH₂CH₂COO⁻ of glutamic acid; —CH₂CH₂C(O)NH₂ of glutamine; —CH₂CH₂CH₂—N—(H)—C(NH₂)⁺—NH₂ of arginine; —CH₂-(imidazole)⁺ group of histidine; and —CH₂(CH₂)₃NH₃⁺ of lysine. Other useful moieties for R¹-R⁴ (and in particular R¹ R²) include alkyls containing 1-6 carbons (straight or branched chains); aralkyls, nucleoside bases and derivatives thereof, carbohydrates and lipids.

There are a number of well known modified forms of the common amino acids such as O-phosphoserine; O-phosphothreonine; O-phosphotyrosine; N-formylmethionine and glycinamide and the side chains of these modified amino acids are also readily used as the R group on the poly NSGs. Typical R-group moieties used in connection with the preferred NSGs are such that the resulting poly NSGs will be biologically active, e.g., mimic or block the activity of a naturally occurring peptide or nonpeptide molecule which adheres to a natural receptor site.

The poly NSGs produced by the process of the invention may be homopolymers, copolymers or interpolymers of any length, i.e., be comprised of a single repeating monomer, two alternating monomer units or which may be randomly and/or wilfully spaced different monomer units. Regardless of the type of poly NSG produced, the poly NSG is produced by the same general procedure which includes repeating a two-step cycle, wherein a new monomer unit is added in each cycle as described in detail below.

The essence of the invention relates to the synthesis process with its repeating two-step cycle. However, some compounds and groups of compounds are also important aspects of the invention. Of specific interest are compounds having the following general structural formula II:

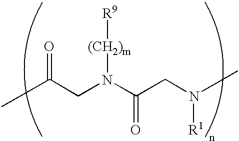

wherein R⁹ is a purine or pyrimidine such as a nucleoside base such as (A, T, G, C or U) or derivative thereof, R¹ is defined above and may be an alkyl moiety containing 1 to 6 carbons, preferably —CH₃ more preferably —H; m is an integer of from 1 to 5 and is preferably 2; and n is an integer of from 1 to 2,000. Compounds of formula II are useful in binding to DNA and RNA and as such can be used as probes and/or in antisense technology. Useful probes can be produced by synthesizing compounds of structural formula II, wherein R⁹ is a nucleoside base, m is 2 and further wherein the monomer units of the compound have the nucleoside bases positioned in a predetermined sequence designed so as to provide for hybridization of the polymer with an appropriate DNA or RNA target. In that the compounds are being used as probes, it is preferable to attach a suitable label to the polymer. Suitable labels are known to those skilled in the art and include radioactive, fluorescent and enzyme labels. Polymers of structural formula II can be used in antisense technology by producing polymers wherein the R⁹ is a purine or pyrimidine base and the sequence of bases in the polymer are designed so as to hybridize to and interrupt the transcription or translation of appropriate DNA and RNA molecules which are known to be pathogenic. When used in connection with antisense technology, the R¹ moiety may be a lipid molecule which would provide for delivery of the compound into the cell and into the nucleus.

Related compounds to compound of formula II are disclosed in Nielsen, P. E., Exholm, M., Berg, R. H. et al. Science, 254 (1991) 1497. By using the synthesis methodology of the present invention the —R¹ moiety can vary to obtain compounds of formula II which have a variety of desirable characteristics such as improved cell penetration with R¹ as a lipid. Further, the R¹ moiety can be used as a site-specific attachment point for a metal chelator, a nuclease, etc.

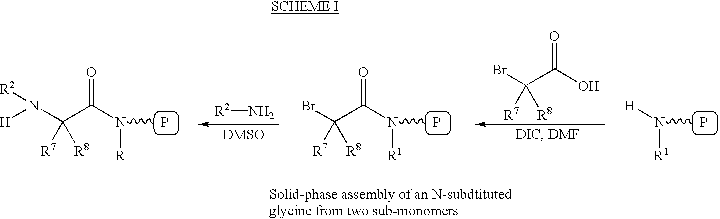

The reaction scheme I includes some abbreviations which refer to reagents used in connection with the invention. For example, DMSO refers to dimethylsulfoxide, DIC refers to N,N-diisopropyl carbodiimide, and DMF refers to N,N-dimethyl formamide. The reaction may be readily carried out at room temperature. However, the reaction may be carried out over a wide range of temperatures between 5° C. and 80° C. Depending on the temperature, the time of the reaction will, of course, vary and can be within the range of 5 minutes to 24 hours. The above temperature, times and reagents are applicable to carrying out the reaction at atmospheric pressure.

In the two-step cycle of the invention shown in Scheme I, amines which are preferably primary amines are bound (using conventional methodology) to a support base surface or solid phase which is represented by the letter “P.” A variety of support resins and connectors to the support resins could be used such as those which are photocleavable, DKP-forming linkers (DKP is diketopiperazine), TFA cleavable, HF cleavable, fluoride ion cleavable, reductively cleavable and base-labile linkers.

The first step of the cycle is the acylation which is carried out by reacting a haloacetic acid such as the bromoacetic acid of Scheme I with the resin-bound secondary amine to obtain an acylated amine.

### Step 1

### Acylation

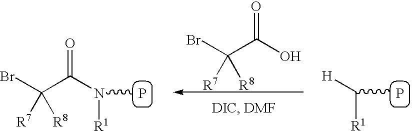

The second step of the cycle is where the side chain or R² group of the monomer unit is added. In the second step, the acylated amine is reacted with an excess of an amine which is preferably a primary amine which includes the R² of group which is to be added at this monomer position in the NSG. The addition of primary amine is preferably done by adding an excess of primary amine which causes a nucleophilic displacement of the leaving group such as a halogen which is the bromine shown in Scheme I. However, any leaving group can be used here provided it is readily removed by nucleophilic displacement, e.g. O-tosyl, O-triflyl, O-mesyl etc.

### Step 2

### Nucleophilic Displacement

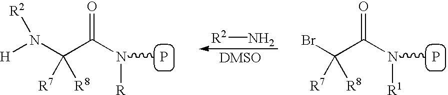

Steps 1 and 2 can be repeated any desired number of times to obtain the desired number of monomer units. In each cycle, step 1 will remain the same. However, in step 2, the R² group of the primary amine can be the same or different as desired to obtain the desired R² group at the desired sequence position in the polymer being produced. The terminal N is shown connected to —R² and H here. However, this is done to allow other cycles to add further monomer units. The actual terminal —N may be capped by providing alkyl and/or acyl groups for R³ and/or R⁴.

Different R groups are correctly positioned in the molecule by using the correct primary amine in step 2 of each cycle. The resulting poly NSG will consist of the desired sequence of monomer units. It is also possible to use the invention to produce mixtures of poly NSGs which mixtures will have known amounts of each poly NSG by reacting (in step 2) mixtures of primary amines with the acylated amine of step 1. By knowing or calculating the reaction rate constant for the reaction of each primary amine with the acylated amine, it is possible to calculate the proportional amounts of each product poly NSG which will result and precisely determine the composition of the resulting mixture of poly NSGs. Such mixtures are useful in that they can be screened to determine which, if any, of the NSGs have a given biological activity, e.g., bind to a known receptor.

Methods of disclosing such mixtures are taught in U.S. Pat. No. 5,010,175 issued Apr. 23, 1991 incorporated herein by reference. Further, the methods of the present invention could be applied in other methods such as that of Houghten, R. A., Proc Natl Acad Sci USA (1985) 82:5131-5135, which teaches a modification of the Merrifield method using individual polyethylene bags. In the general Merrifield method, the C-terminal amino acid of the desired peptide is attached to a solid support, and the peptide chain is formed by sequentially adding amino acid residues, thus extending the chain to the N-terminus. The additions are carried out in sequential steps involving deprotection, attachment of the next amino acid residue in protected form, deprotection of the peptide, attachment of the next protected residue, and so forth.

In the Houghten method, individual polyethylene bags containing C-terminal amino acids bound to solid support can be mixed and matched through the sequential attachment procedures so that, for example, twenty bags containing different C-terminal residues attached to the support can be simultaneously deprotected and treated with the same protected amino acid residue to be next attached, and then recovered and treated uniformly or differently, as desired. The resultant of this is a series of polyethylene bags each containing a different peptide sequence. Although each bag will contain many peptides, all of the peptides in any one bag are the same. The peptides in each bag can then be recovered and individually biologically tested.

The present invention can be used with other methods in order to produce mixtures of poly NSGs which include predetermined amounts of the different poly NSG's in the mixtures including equal molar amounts of each poly NSG in the mixture. The method should be used such that each poly NSG will be present in the mixture in an amount such that it can be retrieved and analyzed. Such mixture of poly NSG's can be generated by synthetic algorithms that involve the splitting of resin beads into equal portions, coupling a unique NSG to each portion and then mixing the portions as described by Furka, A., Sebestyén, M., Asgelom, M. and Dibo, G. (1991) Int. J. Pep. Pro. Res., 37:487-493; Lam, K. et al. (1991) Nature, 354:82-84; Houghten, R. et al. (1991) Nature, 354:84-86; Zuckermann, R. et al. (1991) Patent Appl. PCT WO 91/17823; Zuckermann, R. et al. (1992) Proc. Natl. Acad. Sci. 89:4505-4509 incorporated herein by reference.

The methods of the present invention could also be used in an alternative method deviced by Geysen, H. M., et al., Proc Natl Acad Sci USA (1984) 81:3998-4002.See also WO86/06487 and WO86/00991. This method is a modification of the Merrifield system wherein the C-terminal amino acid residues are bound to solid supports in the form of polyethylene pins and the pins treated individually or collectively in sequence to attach the remaining amino acid residues. Without removing the peptides from support, these peptides can then efficiently be effectively individually assessed for the desired activity, in the case of the Geysen work, interaction with a given antibody or receptor. The Geysen procedure results in considerable gains in efficiency of both the synthesis and testing procedures, while nevertheless producing individual different peptides. The peptides can also be cleaved from the pins and assayed in solution.

Resin bound libraries of poly NSGs can also be prepared by the method of Lam, K. et al. (1991) Nature, 354:82-84, where an equimolar mixture of peptides are synthesized such that each bead contains one unique peptide sequence. A library of beads are then screened for biological activity.

In accordance with other methodology libraries of poly NSGs can be prepared and assayed for biological activity on glass surfaces using light-directed spatially addressable parallel chemical synthesis as described by Fodor, S. et al. (1991) Science, 251:767-773.

Automated Synthesis

The preparation of NSG oligomers by reacting sub-monomers can be adapted to an automated synthesizer (see Zuckermann, R. N., Kerr, J. M., Siani, M. & Banville, S., Int. J. Peptide Protein Res. (1992), in press). Each cycle of monomer addition (as is shown in Scheme I) consists of two steps, (1) an acylation step, and (2) a displacement step—there is no N,α-deprotection step.

The first step, acylation of a resin-bound secondary amine with a haloacetic acid (Lindner, W., Robey, F. A., Int. J. Peptide Protein Res., 30, 794-800 (1987); Robey, F. A., Fields, R. L., Anal. Biochem., 177, 373-377 (1989); Wetzel, R., Halualani, R., Stults, J. T., Quan, C., Bioconjugate Chem., 1, 114-122 (1990)); Fisther, E. Ber. Dtsch. Chem. Ges. (1904), 37:3062-3071 uses a carbodiimide or other suitable carboxylate activation method. A haloacetyl halide could also be used. Acylation of a secondary amine can be difficult, especially when coupling a bulky amino acid. Accordingly, the acylation may be facilitated by the use of haloacetic acids which, in the presence of a carbodiimide, are potent acylating agents.

The second step introduces the side-chain by nucleophilic displacement of the leaving group which is generally a halogen (as a resin-bound α-haloacetamide) with an excess of primary amine. The efficiency of the displacement is modulated by the choice of halide (e.g., I>Cl). Protection of carboxyl, thiol, amino and other reactive side-chain functionalities are desirable to minimize undesired side reactions. However, the mild reactivity of some side-chain moieties toward displacement or acylation may allow their optimal use without protection (e.g., indole, imidazole, phenol).

### EXAMPLES

The following examples are put forth so as to provide those of ordinary skill in the art with a complete disclosure and description of how to carry out the synthesis of the present invention and are not intended to limit the scope of what the inventors regard as their invention. Efforts have been made to ensure accuracy with respect to numbers used (e.g., amounts, temperature, etc.) but some experimental errors and deviation should be accounted for. Unless indicated otherwise, parts are parts by weight, molecular weight is weight average molecular weight, temperature is in degrees Centigrade and pressure is at or near atmospheric.

Oligomer syntheses were performed by an automated synthesizer (Zuckermann, R. N., Kerr, J. M., Siani, M & Banville, S., Int. J. Peptide Protein Res. (1992), in press). The syntheses were conducted with Rink amide polystyrene resin (Rink, H., Tetrahedron Lett., 28, 3787-3790 (1987)) (50 μmol, substitution level 0.45 mmol/g) to avoid diketopiperazine formation. However, a variety of conventional peptide synthesis resins known to those skilled in the art could be used. Acylation reactions were performed by addition of bromoacetic acid (600 μmol, 83 mg) in DMF (0.83 mL), followed by addition of N,N′-diisopropylcarbodiimide (660 μmol, 103 μL) in DMF (170 μL). Reaction mixtures were agitated at room temperature for 30 min. Each acylation was repeated once. Displacement reactions were performed by addition of primary amine (2.0 mmol) as 2.5 M solutions in dimethylsulfoxide (1.0 mL), followed by agitation for 2 hr at room temperature. Optimization of displacement reactions was performed by varying amine concentrations from 0.25 M to 2.5 M. Oligomers were deprotected/cleaved by treatment of the oligomer-resin with 95% trifluoroacetic acid in water (10 mL) for 20 min at room temperature, followed by filtration and lyophilization.

### Examples 1-8

Eight representative penta-NSGs were prepared by the sub-monomer method from a variety of amines, including poorly nucleophilic, sterically-hindered and side-chain protected amines. All compounds were successfully synthesized as established by mass spectrometry, with isolated crude yields between 52 and 90%, and purities generally greater than 85% by HPLC. The purity, yields and mass spectrometry data on the pentamers were obtained and are shown below in Table I.

TABLE I

| | purity | yield | |
|---|---|---|---|
| Oligomer | (%)^{a} | (%)^{b} | MH^{-c} |
| 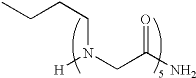 | >85 | 90 | 583.5 |
| 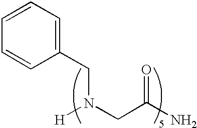 | >85 | 74 | 753.2 |
| 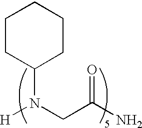 | >85 | 79 | 713.4 |
| 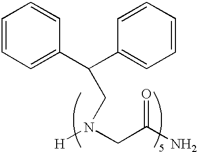 | >85 | 70 | 1204.1 |
| 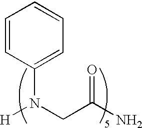 | >85 | 83 | 683.3 |
| 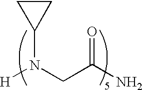 | >85 | 83 | 503.3 |
| 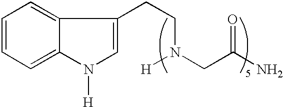 | >60 | 52 | 1018.4 |
| 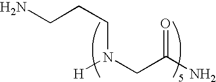 | >85 | 63^{d} | 588.4 |
| 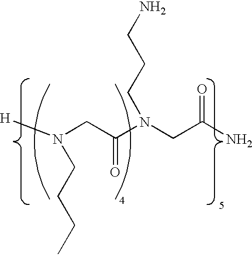 | >65 | 86^{d} | 2850.9 |

^{a}Determined by HPLC.
^{b}Determined from dry weight.
^{c}Liquid-matrix secondary-ion mass spectrometry.
^{d}Made from Boc-NH—CH₂)₃—NH₂.

Optimization of penta-NSG synthesis was performed using combinations of chloro, bromo and iodoacetic acid with both aniline and cyclohexylamine. Bromoacetic acid and iodoacetic acid proved superior to chloroacetic acid in forming penta-(N-phenylglycine) (79%, 83% and <5% yields, respectively). All three haloacetyl compounds successfully gave the penta-(N-cyclohexylglycine) oligomer in >75% yield. However, inclusion of 0.6 M N-hydroxybenzotriazole in the acylation reactions (Robey, F. A., Harris, T. A., Hegaard, N. H. H., Nguyen, A. K., Batinic, D. Chimica Oggi 27-31 (1992)) yielded <5% of the penta-(N-cyclohexylglycine) polymer.

In further optimization studies, the molar concentration of amine was varied from 0.25 M (4.0 equiv.) to 2.5 M (40 equiv.) for n-butylamine, cyclopropylamine and diphenylethylamine using bromoacetic acid. Pentamers were obtained in >80% yield with n-butylamine and cyclopropylamine concentrations >1.0 M, and diphenylethylamine concentrations >2.5 M.

### Example 9

A 25 mer, [(N-n-butylglycine)4(N-(3-amino-propyl)glycine)]₅, was synthesized by the sub-monomer method, thereby demonstrating the utility of this method for the preparation of longer oligomers. Analytical HPLC was performed on a Rainin HPX system controller with a C4 reversed-phase HPLC column (Vydac, 25 cm×4.6 mm) and a gradient elution (solvent A: H20/0.1% TFA and solvent B: CH3CN/0.1% TFA; 10%-75% B in 35 min). Mass spectroscopy confirmed the identity of this compound (MH+=2850.9) which was obtained in 86% yield and 65% purity by HPLC.

The efficient synthesis of a wide variety of oligomeric NSGs using automated synthesis technology, as presented here, makes these polymers attractive candidates for the generation and rapid screening of diverse peptidomimetic libraries.

The instant invention is shown and described herein in what is considered to be the most practical, and preferred embodiments. It is recognized, however, that departures may be made therefrom which are within the scope of the invention, and that obvious modifications will occur to one skilled in the art upon reading this disclosure.

1. A method of synthesizing a poly (N-substituted glycine), comprising the steps of:
acylating an amine resin bound to a substrate to obtain an acylated amine having positioned thereon a leaving group activated toward nucleophilic displacement; and
reacting the acylated amine with a sufficient amount of an amine reactant so as to carry out nucleophilic displacement of the leaving group added during acylation.

2. The method of claim 1, wherein the amine resin bound to the substrate is a secondary amine.

3. The method of claim 1, wherein the amine reactant is a primary amine.

4. The method of claim 1, wherein the leaving group is a halogen.

5. The method of claim 1, wherein the acylating is carried out by reacting the resin-bound amine with a haloacetic acid.

6. The method of claim 5, wherein the halogen atom of the haloacetic acid is selected from the group consisting of Cl, Br, and I.

7. The method of claim 1, further comprising:
sequentially repeating the acylating and reacting steps.

8. A poly (N-substituted glycine) produced by the process of:
acylating a secondary amino resin bound to a substrate to obtain an acylated amine; and
reacting the acylated amine with a sufficient amount of primary amine so as to carry out nucleophilic displacement of a halogen atom added during acylation.

9. The poly (N-substituted glycine) of claim 8 having the following general structural formula I: 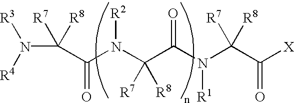
wherein X is —NH₂ or —OH, R¹, R², R³ and R⁴ are independently any molecular moiety attachable to the nitrogen atom, R⁷ and R⁸ are independently any molecular moiety attachable to a carbon atom and n is an integer of from 1 to 2,000.

10. The poly (N-substituted glycine) of claim 9, wherein n is 2 to 100.

11. The poly (N-substituted glycine) of claim 9, wherein R¹, R², R³ and R⁴ are each independently a side chain moiety of a naturally occurring amino acid.

12. The poly (N-substituted glycine) of claim 9 where R⁷ and R⁸ are each —H.

13. A poly (N-substituted glycine) having the following general structural formula II: 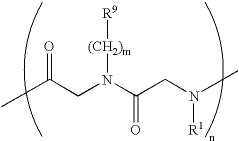
wherein R⁹ is a purine or a pyrimidine or derivative thereof, R¹ is any molecular moiety attachable to a nitrogen atom, m is an integer in the range of from 1-5 and n is an integer within the range of 1 to 2,000.

14. The poly (N-substituted glycine) of claim 13, wherein R⁹ is a nucleoside base, R¹ is a lipid moiety, m is 2 and n in an integer within the range of 3 to 100.

15. The poly (N-substituted glycine) of claim 13, further comprising a detectable label.

16. The poly (N-substituted glycine) of claim 15, wherein the label is a label selected from the group consisting of a radioactive label, a fluorescent label, and an enzyme label.

17. A method of antisense treatment comprising administering to a human a pharmaceutical formulation comprising a pharmaceutically acceptable excipient carrier having dispersed therein a therapeutically effective amount of a compound of structural formula II: 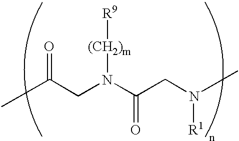
wherein R⁹ is a purine or pyrimidine or derivative thereof, R¹ is any molecular moiety attachable to a nitrogen atom, m is an integer in the range of from 1-5 and n is an integer within the range of 1 to 2,000.
